(19) United States
(12) Patent Application Publication
(10) Pub. No.: US 2011/0311463 A1
(43) Pub. Date: Dec. 22, 2011

(54) HAIR SPRAY AND HAIR MOUSSE WITH NATURAL INGREDIENTS

(76) Inventors: George B. DIAMOND, Glen Gardner, NJ (US); John CARSON, Union City, NJ (US)

(21) Appl. No.: 12/816,990

(22) Filed: Jun. 16, 2010

(51) Int. Cl. A61K 8/04; A61Q 5/06; A61K 8/73

(52) U.S. Cl. 424/47; 424/70.13

(57) ABSTRACT

A naturally occurring film forming material for a hair spray or mousse, rather than a synthetic resin. A preferred naturally occurring film forming material is a polysaccharide, which is a sugar polymer with an INCI name of LEVAN. The film forming material may be mixed with any of a naturally occurring edible oil to add sheen to the hair, naturally occurring substances for antibacterial purposes and a naturally occurring fragrance and an emulsifier.

### BACKGROUND OF THE INVENTION

Hair sprays are fine sprays combining a film forming material which forms a film on the hair and a carrier liquid which is usually volatile and that evaporates leaving the film forming material on the hair. A hair mousse is a foam form of a film forming material and a foam forming agent which also may be volatile and which evaporates leaving the film on the hair.

The film forming material typically used in hair sprays and hair mousses is a synthetic resin. The resin may be dissolved in a carrier comprised of alcohol, water and alcohol mixtures and possibly other volatile mixtures which evaporate leaving the film resin on the hair. The sprays and mousses may contain various other additives, such as silicones, bactericides. emulsifiers and other non-natural, manufactured, synthetic chemicals. They may include herbal products in addition to any synthetic products. They may have synthetic fragrance generating materials. The aerosol version may additionally include synthetic material propellants mixed with the film forming material, hydrofluorocarbons, dimethyl ether as well as natural hydrocarbons that are typically used in aerosol hair sprays.

Many of the synthetic materials used in hair sprays or hair mousses are non-biodegradable materials which may contaminate a water supply and may have harmful effects to anyone or any animal that ingests them. They may also act as severe eye irritants.

The film forming material used herein is LEVAN. There are examples in the prior art of uses of LEVAN in hair care products or hair sprays. Non-naturally occurring resins are used in such LEVAN hair care products, as disclosed in US 2001/0022967, 2006/0029561 and 2008/0102051, whereas the invention hereof does not use such resins. Other uses of LEVAN in hair care products are shown in U.S. Pat. Nos. 7,572,933; 7,179,451; and 7,438,898.

### SUMMARY OF THE INVENTION

The invention hereof uses a natural film forming material for a hair spray or mousse, rather than a synthetic resin. A preferred embodiment of a naturally occurring film forming material is a polysaccharide, which is a sugar polymer with an INCI name of LEVAN.

The resin may be mixed with any of a natural, edible oil to add sheen to the hair, natural substances for antibacterial purposes, a natural fragrance and in some cases a natural substance emulsifier.

### DESCRIPTION OF A PREFERRED EMBODIMENT

LEVAN used in hair spray or mousse, as well as for other uses, is a polymer of fructose, which is fruit sugar, and a synonym for it is Beta 2.6 fructan. LEVAN is a homopolysaccharide which is composed of D-fructofuranosyl residues joined by 2,6 with multiple branches by 2,1 linkages or a group of fructans produced by bacteria or created by breaking down other kinds of plant fructans, called LEVAN beta 2→6.

LEVAN in water or in a water/alcohol solution acts as a film former. Any other additives in the hair spray or hair mousse which includes the LEVAN are preferably natural rather than synthetic substances.

LEVAN is subject to bacterial action. LEVAN used in natural products should be protected from bacterial action. In the invention, that may be done with natural herbicides or bactericides. A bactericide should be included for product safety and natural ingredients. A herbal one is preferably used in this case. Examples of natural bactericides include an extract from one of:
Organum Vulgare Leaf Extract
Thymus Vulgaris (Thyme) Extract
Cinnamonium Zylanicum Bark Extract (Cinnamon)
Rosemarious Officialis (Rosemary) Leaf Extract
Lavandula Anstixolia (Lavender) Extract
Citrus Medica Liminium (Lemon Peel) Extract
Mentha Pepperita (Peppermint Leaf) Extract
Adrasti Canardensis (Golden Seal Root) Extract
Olea Europaea (Olive Leaf) Extract

To perhaps provide additional sheen to the hair, natural edible oils are added, such as grape seed oil, flax seed oil, etc. For antibacterial and preservation purposes, a combination of herbal extracts may be used. A natural fragrance may be added, rather than a synthetic fragrance. Some hair sprays and mousse require an emulsifier for sufficient mixing of the ingredients. A natural emulsifier may be used. An example may be a licorice extract.

For the aerosol spray version of a hair spray, a propellant used would be a natural liquefied or gaseous form hydrocarbon alone or combined with naturally occurring compressed gases, such as carbon dioxide, nitrogen, argon, etc. or a naturally occurring compressed gas, can by used by itself.

In a hair spray or mousse of all naturally occurring ingredients, trace amounts of other ingredients may be present. Therefore, the present invention consists essentially of the described ingredients.

Depending on the desired level of hold for the hair spray, normal, extra hold or a maximum hold, the amount of LEVAN by weight in the mixture with other ingredients varies in the range of approximately 2% to 12%. The percentage by weight of other major ingredients in the mixture depends on regulations of a local jurisdiction, such as state or country, regarding the total amount of a volatile organic substances (voc) permitted in a sprayable product.

If herbal or botanical ingredients are used, depending upon the type of such ingredients, their weight range usually is from 0.1 to 0.5%. If edible oils are used, their weight range in the final mixture varies from 0.1 to 2%. When a propellant is used, it varies by weight in the range of 2% to 30%.

The mixture, appropriately adjusted to be dispensed, can be used in a pump spray or aerosol spray version to be a hair spray or a hair mousse. The hair mousse comes out as foam, while the spray comes out as a liquid spray.

All of the above mentioned ingredients are natural, not synthetically or non-naturally occurring ingredients. It is possible that trace amounts of non-naturally occurring ingredients may be mixed into the mixture being dispensed. That possibility is considered as it is noted herein that the hair spray or mousse consists essentially of the named ingredients.

Although the present invention has been described in relation to particular embodiments thereof, many other variations and modifications and other uses will become apparent to those skilled in the art. It is preferred, therefore, that the present invention be limited not by the specific disclosure herein, but only by the appended claims.

What is claimed is:

1. A hair spray or hair mousse consisting essentially of naturally occurring ingredients including a film forming material including a natural film forming agent polysaccharide LEVAN and a water or water and alcohol mixture carrying the LEVAN.

2. The hair spray or hair mousse of claim 1, further consisting essentially of additional naturally occurring ingredients for at least one of antibacterial and fragrance providing purposes.

3. The hair spray or hair mousse of claim 2, further comprising a naturally occurring emulsifier.

4. The hair spray or hair mousse of claim 1, further comprising a naturally occurring emulsifier.

5. The hair spray or hair mousse of claim 1, mixed with a naturally occurring liquefied and/or gaseous propellant to facilitate spraying of the hair spray or hair mousse in spray or mousse form.
